(19) United States
(12) Patent Application Publication
(10) Pub. No.: US 2010/0266520 A1
(43) Pub. Date: Oct. 21, 2010

(54) COSMETIC COMPOSITION COMPRISING A SEMICRYSTALLINE POLYURETHANE/POLYESTER COPOLYMER

(76) Inventors: Stephane Arditty, Ballainvilliers (FR); Jean Mondet, Aulnay-sous-Bois (FR)

Correspondence Address: OLIFF & BERRIDGE, PLC, P.O. BOX 320850, ALEXANDRIA, VA, 22320-4850, US

(21) Appl. No.: 12/741,519

(22) PCT Filed: Dec. 17, 2008
(86) PCT No.: PCT/IB08/55389

§ 371 Date: Jun. 28, 2010

Related U.S. Application Data
(60) Provisional application No. 61/006,596, filed on Jan. 23, 2008.

(30) Foreign Application Priority Data
Dec. 17, 2007 (FR) 07559892

(51) Int. Cl. A61K 8/87; A61K 8/72; A61K 8/85; A61Q 5/00; A61Q 1/10; B65D 79/00

(52) U.S. Cl. 424/70.11; 206/216

(57) ABSTRACT

The present invention relates to a cosmetic composition including, in a physiologically acceptable medium, an effective quantity of at least one semicrystalline polyurethane/polyester copolymer and at least one dye substance, the copolymer having a melting point greater than or equal to 20° C.

The subject of the present invention is a cosmetic composition endowed with thermally modulatable mechanical properties. The subject of the invention is also a method for making up and/or caring for keratin fibers.

The composition according to the invention may be more particularly a cosmetic composition for coating keratin fibers such as the eyelashes, the eyebrows and the hair of human beings, or alternatively false eyelashes.

Thus, the composition according to the invention may be a makeup composition, also called mascara, a makeup base for keratin fibers or also termed base coat, a composition to be applied on a makeup, also termed top coat, or alternatively a composition for treating keratin fibers. More especially, the composition according to the invention is a mascara.

Curling is (with lengthening, volume, separation) one of the main expectations expressed by the consumer in relation to their mascara. For them, the curling is expressed through shaping of the eyelashes, but also and especially the stability of this curl over several days. This expectation, which is partly linked to the typology of the eyelashes, is in particular very clearly expressed in Asia.

So-called curling mascara compositions are thus already known from the prior art which comprise a mixture of waxes and a film-forming polymer such as the compositions described in the document EP-B-0928607.

The use of organogelling agents in mascara compositions in order to replace all or some of the waxes in order to obtain improved properties for curling of the eyelashes is also known from the document WO 00/74519. It is to be noted that the homopolymer of urethane available from BASF under the tradename Luviset® P.U.R. is not a semicrystalline copolymer.

However, such compositions do not allow optimum curling of the eyelashes.

On the other hand, devices exist for curling the eyelashes, such as “eyelash curlers”. One type of eyelash curler consists, for example, in gripping the eyelashes between the jaws of tongs in order to confer on them a curled shape before the making up, but this operation is delicate to carry out. Other eyelash curlers are in the form of heating tongs or brush, as described in the documents U.S. Pat. No. 5,853,010 or JP 2000-38314, for shaping the eyelash under the action of heat. In fact, these heating instruments often offer an insufficient curling effect.

In fact, the possibility of shaping the eyelash by taking advantage of a sufficient period favorable to its handling, and of a good resistance of this shaping over time involves antagonist properties of the deposit. In a first instance, it is necessary to be able to work the deposit when it is malleable, but in a second instance, it is imperative that this deposit has good mechanical properties in order to durably maintain the eyelashes in their curled position.

To satisfy these two requirements, it has more recently been proposed to exploit, in mascara-type compositions, materials which change state under the effect of a stimulus such as heat.

Thus, the documents US 2005/0031656 and US 2005/0058496 propose compositions which use, for these purposes, a combination of an amorphous film-forming polymer and a compound with a heat profile, chosen in particular from waxes, semicrystalline polymers and thickened oils.

Surprisingly, the inventors have discovered that it was now possible to further optimize this curling effect by exploiting the ability of compounds, different from those mentioned above, to undergo modification of their mechanical properties resulting in particular in a rigidification in response to a rise in temperature.

More specifically, the subject of the invention is a care and/or makeup cosmetic composition for keratin fibers comprising, in a physiologically acceptable medium, an effective quantity of at least one semicrystalline polyurethane/polyester copolymer and at least one dye substance, said copolymer having a melting point greater than or equal to 20° C.

The expression “physiologically acceptable medium” is understood to mean a nontoxic medium, which is capable of being applied to keratin fibers, such as the eyelashes, the eyebrows and the hair of human beings, and which is in particular compatible with the ocular region.

The subject of the present invention is also the use of a cosmetic composition as defined above to obtain a film deposited on keratin fibers having improved curling properties.

The subject of the invention is also a cosmetic method for making up or for the nontherapeutic care of the keratin fibers comprising the application, to the keratin fibers, of a cosmetic composition in accordance with the invention, said composition being, prior to, simultaneously with or subsequent to its application, heated to a temperature greater than or equal to the melting point of the copolymer which it contains.

The composition may be heated to such a temperature prior to, simultaneously with or subsequent to its application, in particular with the aid of an application device comprising heating means, such as a heating brush.

Thus, the compositions in accordance with the invention have the characteristic feature, when they are heated to a temperature greater than the melting point of the copolymer which they contain, of leading to the formation, at the surface of the keratin fibers, of a homogeneous film having very satisfactory curling properties.

Thus, it is possible to shape the eyelashes coated with such a composition under the action of a source of heat (such as a heating brush) having a temperature greater than or equal to the melting point of the copolymer, and rapidly.

### Semicrystalline Polyurethane/Polyester Copolymer

The expression “crystallizable polyurethane/polyester copolymer” is understood to mean a polyurethane polymer containing at least one crystallizable polyester block. This or these polyester block(s) may be one or more polyester blocks which are either directly present inside the polyurethane chain forming the backbone or forming one or more grafts linked to this polyurethane chain, it being possible for these two options to also exist simultaneously at the level of the polyurethane.

The expression “semicrystalline polyester” is understood to mean a polyester having a chain or crystallizable chain parts which have a melting temperature which causes a change of phase of the polymer (solid-liquid transition).

The expression “crystallizable chain or block” is understood to mean, for the purposes of the invention, a chain or block which, if it were alone, would pass from the amorphous state to the crystalline state, irreversibly, depending on whether the temperature is above or below this melting temperature.

The expression “block” is generally understood to mean a grouping of atoms belonging to the backbone, a grouping constituting one of the repeating units of the polymer. A block generally comprises at least 5 identical repeating units. The crystallizable block(s) is (are) therefore of a chemical nature which is different from the amorphous block(s). When the crystallizable part is in the form of a crystallizable block of the polymeric backbone, the amorphous part of the polymer is in the form of an amorphous block; the semicrystalline polymer is in this case a block copolymer, for example of the diblock, triblock or multiblock type, containing at least one crystallizable block and at least one amorphous block.

The expression “chain” is understood to mean, for the purposes of the invention, a pendent or side grouping of atoms relative to the polymer backbone. Advantageously, the “crystallizable pendent chain” may be a chain containing at least 6 carbon atoms. When the polymers of the invention are polymers with crystallizable side chains, they are advantageously in a random form.

The semicrystalline polyurethane/polyester copolymers which can be used in the composition according to the invention have a melting point greater than or equal to 20° C., in particular ranging from 25° C. to 100° C., preferably ranging from 30° C. to 80° C. and even better from 35° C. to 70° C.

The melting point may be measured by any known method and in particular with the aid of a differential scanning calorimeter (D.S.C), for example the calorimeter sold under the name DSC 30 by the company METLER, according to the protocol as described below.

A sample of 5 to 10 mg of copolymer placed in a crucible is subjected to a first rise in temperature ranging from −20° C. to 110° C., at the heating rate of 5° C./minute, and is then cooled from 110° C. to −20° C. at a cooling rate of 5° C./minute and finally subjected to a second rise in temperature ranging from −20° C. to 110° C. at a heating rate of 5° C./minute. During the second rise in temperature, the variation of the difference between the power absorbed by the empty crucible and the crucible containing the copolymer sample is measured as a function of time.

The value of the melting point corresponds to the peak maximum of the thermal profile thus obtained.

Advantageously, the semicrystalline copolymer(s) used according to the invention have a number-average molecular mass Mn greater than or equal to 1000.

Advantageously, the semicrystalline copolymer(s) of the composition of the invention have a number-average molecular mass M̅n ranging from 2000 to 800 000, preferably from 3000 to 500 000, even better from 4000 to 150 000, in particular less then 100 000, and even better from 4000 to 99 000.

Preferably, the crystallizable block(s) or chain(s) of the copolymers according to the invention represent at least 20% of the total weight of the copolymer and even better at least 30%.

The semicrystalline polyurethane/polyester copolymers which can be used in the composition according to the invention are of synthetic origin.

A composition in accordance with the present invention may contain from 1 to 60% by weight, in particular from 3 to 50% by weight, and more particularly from 5 to 40% by weight of semicrystalline polyurethane/polyester copolymer(s).

In general, the polyurethane/polyester copolymers considered in the invention are polymers formed by a reaction between at least one diisocyanate derivative and a semicrystalline polyester, having a reactive —OH or —NH₂ group, preferably an —OH group, at each alpha or omega end of its chain, specifically aimed at giving it, in the context of the present invention, thermovariable mechanical properties.

More specifically, the semicrystalline polyurethane/polyester copolymers according to the invention may be obtained by the reaction between at least one crystalline or semicrystalline aliphatic polyester, with alpha- and omega-OH ends and at least one aliphatic, cycloaliphatic or aromatic, generally C₃-C₅₀, diisocyanate derivative.

According to a simplified embodiment, the polyester and diisocyanate considered may be brought into contact in equimolar quantities and under conditions favorable to their respective interaction, so as to form the expected copolymer.

According to another variant embodiment, which is in particular preferred in the context of the present invention, this reaction between these two compounds may be carried out in the presence of at least a third type of compound termed coupling agent or coupler.

As described in greater detail below, this compound may be a coupler of diol, diamine or amino alcohol nature.

According to this variant, the structure of the polyurethane/polyester copolymer is then formed of blocks, alternating at least two different types of segment, termed hard segments and soft segments, linked together by covalent linkages. The so-called hard segments are polyurethane (and/or polyurea) segments formed by the reaction of the diisocyanates and the coupler. They have a high density of a urethane group of high polarity and are rigid at room temperature. As regards the soft segments, they are segments formed by the reaction between the diisocyanate and the polyester. They have a low polarity which is that of the polyester. Since they have a low density of urethane units, they are flexible at room temperature and are therefore responsible for the characteristics of elongation of the composition containing them. What is more, in the light of the chemical nature forming the polyester, they are crystallizable when they are exposed to a temperature thus conferring on the composition containing them the rigidity expected upon their cooling.

Aliphatic, Cycloaliphatic and/or Aromatic Diisocyanates

As regards the aliphatic, cycloaliphatic and/or aromatic C₃ to C₅₀ diisocyanates, they are well known to a person skilled in the art and are commercially available.

Thus, suitable for the invention are the diisocyanates of formula

OCN—R₅—NCO

in which R₅ is a divalent group chosen from alkylene, arylene, cycloalkylene, alkarylene or arylalkylene groups, said groups being linear or branched and comprising from 1 to 50 carbon atoms and it being possible for said groups to comprise at least one oxygen, sulfur and/or nitrogen atom.

More particularly, R₅ may be chosen from the following groups:

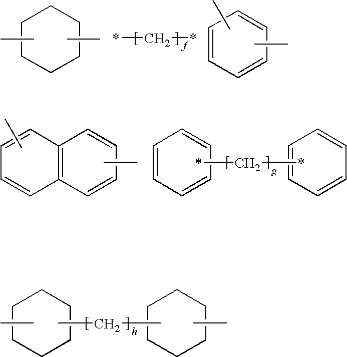

With:

f being an integer ranging from 1 to 30,

g being an integer ranging from 1 to 3, and

h being an integer ranging from 1 to 20, preferably from 1 to 12.

Preferably, R₅ is chosen from the divalent radicals hexamethylene, isophorone, 2,4-toluene, 2,6-toluene, 4,4′-dicyclohexylmethane, para-phenylene, and 1,5-naphthylene.

Most particularly suitable are toluene diisocyanate (TDI), 4,4′-diphenylmethane diisocyanate (MDI), naphthalene diisocyanate, phenylene diisocyanate, xylene diisocyanate (XDI), tetramethylene xylene diisocyanate (TMXDI), isophorone diisocyanate (IPDI), 4,4′-dicyclohexylmethane diisocyanate (HMDI) and hexamethylene diisocyanate (HDI).

Advantageously, the copolymer according to the invention is derived from the reaction of at least one polycaprolactone with at least 4,4′-diphenylmethane diisocyanate (MDI) or isophorone diisocyanate (IPDI).

Crystallizable Polyester

As specified above, the copolymer in accordance with the present invention has at least one semicrystalline polyester block.

The quantity of these blocks is adjusted in order to allow said copolymer to manifest the expected melting point according to the invention.

These polyester blocks are more particularly aliphatic polyesters.

As is evident from the detailed description presented below, these polyester units may be characterized according to their method of preparation.

According to a first variant, they may result from the polyaddition of a C₃₋₂₀ lactone that is substituted or unsubstituted, preferably unsubstituted, with a C₁₋₆ alkyl group.

According to a second variant, they may be obtained by polycondensation of at least one linear, branched or cyclic, preferably linear, C₄₋₅₀ aliphatic diacid and at least one linear, substituted or cyclic, preferably linear, aliphatic C₄-C₅₀ diol.

According to a third variant, they may be derived from the transesterification and polycondensation between at least one C₄-C₅₀ diol as defined above and a dimethyl or diethyl diester of a C₄-C₅₀ dicarboxylic acid, also as defined above.

These three alternatives are more particularly discussed below.

1) Polyesters Obtained by Opening of a Lactone Ring or Cyclic Esters

These polyesters are polyesters obtained by polyaddition of a lactone, generally as C₃₋₂₀, that is substituted or unsubstituted with a C₁₋₆ alkyl group, initiated by a diol.

The C₃₋₂₀ lactones are cyclic monoesters of general formula:

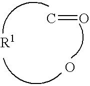

with —R₁— representing a linear or branched C₂₋₁₉ alkylene unit of which one or more H atoms are substituted or unsubstituted.

Reference may be made for example to the document WO-A-0042091 by DSM page 13.

As representative of unsubstituted lactones, there may be mentioned in particular β-propiolactone; β-butyrolactone; β-valerolactone; δ-valerolactone; δ-caprolactone; ε-caprolactone; ω-enantholactone; ω-caprylolactone; ω-laurolactone.

As regards substituted lactones, they are substituted on the C atoms of the ring of which one or more H atoms may be substituted by a C₁₋₂₂ alkyl group. This alkyl group may itself be linear or branched or contain a ring. By way of illustration of these substituted polylactones, there may be mentioned in particular β-methyl-δ-valerolactone and α-methyl-ε-caprolactone.

Among the lactones cited, ε-caprolactone is preferred. It corresponds to the C₃-C₂₀ lactone as defined above wherein R₁—(CH₂)₅ group.

Also suitable are the cyclic esters which are no longer monoesters but diesters and which correspond, for example, to the following formula:

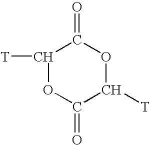

in which T represents hydrogen or a C_{1 to 10} alkyl group.

If T represents CH₃, the compound is lactide.

If T represents H, the compound is glycolide.

Taking various optical isomers: the following compounds can be identified: L,L-lactide; D,D-lactide; D,L-lactide. It should be noted that lactones and lactides can copolymerize together.

Among these cyclic esters, ε-caprolactone is most particularly suitable.

As specified above, the opening of a ring as defined above, leading to a polycaprolactone with —OH ends, is generally initiated with a diol.

According to a particular embodiment, the crystallisable polyester according to the invention is obtained by opening a ε-caprolactone.

For the synthesis of this type of polyester, reference may be made in particular to the protocol described in the document EP 1 457 514.

2) Diol polyesters obtained by polycondensation (or transesterification) between at least one linear, branched or cyclic, preferably linear, C₄-C₅₀ dicarboxylic acid (or its methyl or ethyl diester in the case of transesterification) and at least one aliphatic, linear, substituted or cyclic, preferably linear C₄-C₅₀ diol.

Regardless of the reaction considered, namely polycondensation or transesterification, the reaction is carried out in the presence of an excess of diol in relation to the stoichiometry of the diacid or diester so that the polycondensate carries a hydroxyl functional group at each of its ends.

a) Aliphatic or Cycloaliphatic Dicarboxylic Acid and their Derivatives

For the purposes of the invention, the dicarboxylic acid derivatives cover in particular their acid dichloride, their anhydride derivatives or their methyl or ethyl diesters.

These diacids may in particular be represented by the formula HOOC—R₃—COOH, where R₃ represents a group chosen from linear, branched or cyclic C₂₋₅₀ divalent hydrocarbon groups such as saturated or unsaturated alkylene or arylene, preferably alkylene, groups which may contain in their chain one or more heteroatoms such as O, N, S and/or Si, —and their acid chloride and acid anhydride derivatives. Among these dicarboxylic acids, there may be mentioned more particularly succinic, glutaric, adipic, pimelic, azelaic, sebacic, suberic, itaconic, cyclohexanedicarboxylic and dodecanedicarboxylic acids which may also be used in the form of their derivatives as specified above.

According to one variant embodiment, the reaction medium may additionally contain at least one required aliphatic carboxylic acid, an aromatic diacid such as the isomers of terephthalic acid for example, provided that the polymer thus obtained is semicrystalline suitable for the present invention.

b) Aliphatic or Cycloaliphatic Diol

The aliphatic diols suitable for the invention may correspond in particular to the formula

HO—R₂—OH,

where R₂ represents a group chosen from linear, branched or cyclic C₂₋₅₀ divalent hydrocarbon groups such as saturated or unsaturated alkylene groups which may contain in their chain one or more heteroatoms such as O, N, S and/or Si and which may be partially or totally substituted with fluorine atoms.

In the case of the present invention, most particularly suitable in this regard are ethylene glycol, propylene glycol, diethylene glycol, neopentyl glycol, 1,3-butanediol, 1,3-propanediol, 1,4-butanediol, 1,5-pentanediol, 1,6-hexanediol, 1,6-cyclohexanediol, 1,10-decanediol, 1,12-dodecanediol, dipropylene glycol, furandimethanol, cyclohexanedimethanol, and polyols such as glycerol and sorbitol, and mixtures thereof.

The diol polyesters according to the invention may also be obtained by polycondensation of a hydroxy acid.

By way of illustration of these hydroxy acids, there may be mentioned in particular alpha-, beta-, gamma- and omega-hydroxycarboxylic acids such as for example lactic acid, malic acid and citric acid.

In addition to the abovementioned two classes of polyesters which are most particularly suitable for the invention, the following categories of polyesters may also be mentioned:

3) Diol Polyesters Resulting from Bacterial Polymerization

By way of representatives of these natural polyesters, there may be mentioned in particular poly(beta-hydroxyalkanoates) such as polyhydroxybutyrate (PHB) and more particularly the poly-3-hydroxybutyrate form, but also poly-4-hydroxybutyrate, polyhydroxyvalerate (PHV), polyhydroxyhexanoate (PHH), polyhydroxyoctanoate (PHO) and copolymers thereof.

4) Diol Polycarbonates Corresponding to the General Formula

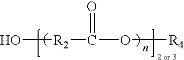

in which R₂ represents a C₄-C₅₀ diol, in particular as defined above, R₄ is a linear or branched, optionally substituted or trivalent, alkyl, alkenyl or alkynyl radical preferably having 1 to 20 carbon atoms and n is selected such that the molecular mass of the diol polycarbonates is between about 300 and about 10 000 g/mol, preferably between about 300 and about 5000 g/mol and more preferably still between about 300 and about 2000 g/mol.

These diol polycarbonates have the advantage of being semicrystalline and of having a melting point in accordance with the requirements that are needed in the context of the present invention.

The choice of the materials necessary for the preparation of these polyesters is made taking into account this requirement. This aspect is clearly within the capacity of persons skilled in the art.

In the event that it would however be desired to prefer the use of a compound not able as such to adjust the melting point of the polyester corresponding to this temperature range, it remains possible to combine with this polyester another type of polymer block, which is this time not crystallizable, so as to precisely reduce the melting point to an expected value, for example a polyamide block or a polydiene block or a silicone block.

As specified above, the semicrystalline polyurethane/polyester copolymers may be obtained according to a preferred variant by reacting the diisocyanate, in particular as defined above, and at least one polyester as defined above, in the presence of a coupling agent.

Coupling Agents or Couplers

The couplers suitable for the invention may be diols, diamine derivatives or amino alcohol derivatives, in particular of the following formulae:

HO—R₆—OH,

H₂N—R₆—NH₂ or

HO—R₆—NH₂

in which R₆ represents a saturated or unsaturated, linear or branched alkylene group comprising from 1 to 50 carbon atoms, an arylene group comprising from 6 to 50 carbon atoms, a cycloalkylene, alkarylene or arylalkylene group, said groups being linear or branched and comprising from 6 to 50 carbon atoms.

By way of illustration of the low-molecular weight diols, the following compounds may be more particularly mentioned:

1,2-butanediol, 1,2-pentanediol; 4-methyl-1,2-pentanediol, 2-methyl-1,2-pentanediol, 3,3-methyl-1,2-butanediol, 4-methyl-1,2-hexanediol; 1,2-heptanediol, 3-phenyl-1,2-propanediol; 1,2,6-hexanetriol, 1,2-hexanediol, 1,2,4-butanetriol and combinations thereof.

According to one variant embodiment, the reaction between the diisocyanate derivative and the polyester may be carried out in the presence of a coupler bearing an ionizable functional group, for example of the carboxylic, sulfonic or amine type, more particularly of the carboxylic type.

Such a coupler may in particular be advantageous for the purpose of isolating the copolymer obtained after preparation.

By way of illustration of such couplers, dimethylolpropionic acid may in particular be mentioned.

According to yet another variant embodiment, the reaction between the isocyanate derivative and the polyester derivative may be carried out in the presence of two types of coupler, one carrying at least one ionizable functional group.

The reaction conditions for preparing the polyurethane polyester copolymer which are considered in the context of the present invention involve procedures which are clearly within the competence of persons skilled in the art and are not specified below. Such a method is described in particular in patent EP 1 457 914.

The category of polyester derived from lactones is particularly advantageous in the context of the present invention since these polymers generally have a melting temperature limited to and less than 60° C., and a rapid rate of crystallization, allowing the properties expected for the desired shaping in the context of the present invention to be rapidly obtained.

By way of illustration of the copolymers most particularly suitable for the invention, there may be mentioned in particular those derived from the reaction between the polyester of the polycaprolactone type, in particular having a molecular weight varying from 1000 to 6000 and in particular from 2000 to 5500 and an oligoisocyanate, chosen in particular from 4,4′-di-phenylmethane diisocyanate (MDI) and isophorone diisocyanate (IPDI), in the presence or absence of a coupler, such as for example 1,4-butanediol.

There may be mentioned more particularly by way of example of this type of copolymer, semicrystalline polyurethane/polycaprolactone copolymers. More particularly the composition according to the invention may comprise those semicrystalline polyurethane/polycaprolactone copolymers marketed under the references Tecoflex® CLA-60D-V and Tecoflex® CLA-93A-V resin by Noveon, or those marketed by Merquinsa under the references Pearlbond® and Disperbond®, in particular Disperbond® D31S and Disperbond® D39S, and mixtures thereof.

Dye Substances

The composition according to the invention comprises a dye substance such as pulverulent dye substances, fat-soluble dyes, water-soluble dyes. This dye substance is present in an amount ranging from 0.1% to 20% by weight, relative to the total weight of the composition, preferably ranging from 1% to 15% by weight.

The pulverulent dye substances may be chosen from pigments and pearlescent agents.

The pigments may be white or colored, inorganic and/or organic, coated or uncoated. There may be mentioned, among the inorganic pigments, titanium dioxide, optionally surface-treated, zirconium, zinc or cerium oxides, and iron or chromium oxides, manganese violet, ultramarine blue, chromium hydrate and ferric blue. Among the organic pigments, there may be mentioned carbon black, the D & C type pigments, and lacquers based on carmine cochineal, barium, strontium, calcium or aluminum.

The pearlescent agents may be chosen from white pearlescent pigments such as mica coated with titanium or bismuth oxychloride, colored pearlescent pigments such as mica-titanium with iron oxides, mica-titanium with in particular ferric blue or chromium oxide, mica-titanium with an organic pigment of the abovementioned type and pearlescent pigments based on bismuth oxychloride.

The fat-soluble dyes are for example Sudan Red, D & C Red 17®, D & C Green 6®, β-carotene, soybean oil, Sudan Brown, D & C Yellow 11®, D& C Violet 2®, D& C Orange 5®, quinoline yellow, annatto. The water-soluble dyes are for example beet juice and methylene blue.

Physiologically Acceptable Medium

The composition according to the invention may be provided in the form of a continuous aqueous phase or in anhydrous form or in the form of a water-in-oil emulsion, oil-in-water emulsion, water-in-oil dispersion or oil-in-water dispersion. It may be solid, liquid or pasty.

The composition according to the invention may thus comprise an aqueous phase which may consist mainly of water. It may also comprise a mixture of water and of a water-miscible solvent such as lower monoalcohols having from 1 to 5 carbon atoms such as ethanol, isopropanol, glycols having from 2 to 8 carbon atoms such as propylene glycol, ethylene glycol, 1,3-butylene glycol, dipropylene glycol, C₃-C₄ ketones, C₂-C₄ aldehydes.

The aqueous phase (water and optionally water-miscible organic solvent) may represent from 5% to 95% by weight, relative to the total weight of the composition.

The composition generally comprises a fatty phase. This comprises at least one oil and/or at least one wax.

The expression “oil” is understood to mean a fatty substance which is liquid at room temperature (25° C.) and atmospheric pressure (760 mmHg, that is 105 Pa). The oil may be volatile or nonvolatile.

The expression “volatile oil” is understood to mean, for the purposes of the invention, an oil capable of evaporating upon contact with the skin or the keratin fiber within less than one hour, at room temperature and atmospheric pressure. The volatile oils of the invention are volatile cosmetic oils, that are liquid at room temperature, having a nonzero vapor pressure, at room temperature and atmospheric pressure, ranging in particular from 0.13 Pa to 40 000 Pa (10⁻³ to 300 mmHg), in particular ranging from 1.3 Pa to 13 000 Pa (0.01 to 100 mmHg), and more particularly ranging from 1.3 Pa to 1300 Pa (0.01 to 10 mmHg).

The expression “nonvolatile oil” is understood to mean an oil that remains on the skin or the keratin fiber at room temperature and atmospheric pressure for at least several hours and has in particular a vapor pressure of less than 10⁻³ mmHg (0.13 Pa).

The oil may be chosen from any physiologically acceptable and in particular cosmetically acceptable oils, in particular mineral, animal, vegetable and synthetic oils; in particular volatile or nonvolatile hydrocarbon and/or silicone and/or fluorinated oils and mixtures thereof. More precisely, the expression “hydrocarbon oil” is understood to mean an oil mainly comprising carbon and hydrogen atoms and optionally one or more functional groups chosen from hydroxyl, ester, ether, and carboxyl functional groups. Generally, the oil has a viscosity of 0.5 to 100 000 mPa·s, preferably from 50 to 50 000 mPa·s and preferably still from 100 to 300 000 mPa·s.

By way of example of a volatile oil which can be used in the invention, there may be mentioned:
the volatile hydrocarbon oils chosen from hydrocarbon oils having from 8 to 16 carbon atoms, and in particular branched C₈-C₁₆ alkanes such as C₈-C₁₆ isoalkanes of petroleum origin (also called isoparaffins) such as isododecane (also called 2,2,4,4,6-pentamethylheptane), isodecane, isohexadecane, and for example the oils sold under the trade names Isopars® or Permethyls®, the branched C₈-C₁₆ esters isohexyl neopentanoate, and mixtures thereof. Other volatile hydrocarbon oils such as petroleum distillates, in particular those sold under the name Shell Solt® by the company SHELL, may also be used;
volatile silicones, such as for example volatile linear or cyclic silicone oils, in particular those having a viscosity ≦8 centistokes (8×10⁻⁶ m²/s), and having in particular from 2 to 7 silicon atoms, these silicones optionally comprising alkyl or alkoxy groups having from 1 to 10 carbon atoms. As volatile silicone oil which may be used in the invention, there may be mentioned in particular octamethyl cyclotetrasiloxane, decamethyl cyclopentasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane and mixtures thereof.

There may also be mentioned the volatile linear alkyltrisiloxane oils of general formula (I):

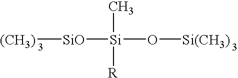

where R represents an alkyl group comprising from 2 to 4 carbon atoms and in which one or more hydrogen atoms may be substituted with a fluorine or chlorine atom.
Among the oils of general formula (I), there may be mentioned: 3-butyl 1,1,1,3,5,5,5-heptamethyl trisiloxane, 3-propyl 1,1,1,3,5,5,5-heptamethyl trisiloxane, and 3-ethyl 1,1,1,3,5,5,5-heptamethyl trisiloxane, corresponding to the oils of formula (I) for which R is respectively a butyl group, a propyl group or an ethyl group.

By way of example of a nonvolatile oil which can be used in the invention, there may be mentioned:
hydrocarbon oils of animal origin such as perhydrosqualene;
hydrocarbon vegetable oils such as the liquid triglycerides of fatty acids of 4 to 24 carbon atoms, such as the triglycerides of heptanoic or octanoic acids or alternatively wheatgerm oil, olive oil, sweet almond oil, palm oil, rapeseed oil, cottonseed oil, alfalfa oil, poppy seed oil, pumpkin seed oil, gourd oil, blackcurrant seed oil, evening primrose oil, millet oil, barley oil, quinoa oil, rye oil, safflower oil, candlenut oil, passionflower oil, musk rose oil, sunflower oil, corn oil, soybean oil, grapeseed oil, sesame oil, hazelnut oil, apricot oil, macadamia oil, castor oil, avocado oil, the triglycerides of caprylic/capric acids such as those sold by the company Stearineries Dubois or those sold under the names Miglyol® 810, 812 and 818 by the company Dynamit Nobel, jojoba oil, Shea butter; linear or branched hydrocarbons of inorganic or synthetic origin such as paraffin oils and their derivatives, petroleum jelly, polydecenes, polybutenes, hydrogenated polyisobutene such as Parleam®, squalane;
synthetic ethers having from 10 to 40 carbon atoms;
synthetic esters in particular of fatty acids such as the oils of formula R₁COOR₂ in which R₁ represents the residue of a linear or branched higher fatty acid comprising from 1 to 40 carbon atoms and R₂ represents a hydrocarbon chain, which is in particular branched, containing from 1 to carbon atoms with R₁+R₂≧10 such as for example purcellin oil (cetostearyl octanoate), isononyl isononanoate, isopropyl myristate, isopropyl palmitate, C₁₂ to C₁₅ alcohol benzoate, hexyl laurate, diisopropyl adipate, isononyl isononanoate, 2-ethylhexyl palmitate, 2-octyldodecyl stearate, 2-octyldodecyl erucate, isostearyl isostearate, tridecyl trimellitate; octanoates, decanoates or ricinoleates of alcohols or polyalcohols such as propylene glycol dioctanoate; hydroxylated esters such as isostearyl lactate, octyl hydroxystearate, octyldodecyl hydroxystearate, diisostearyl malate, triisocetyl citrate, heptanoates, octanoates, decanoates of fatty alcohols; polyol esters such as propylene glycol dioctanoate, neopentyl glycol diheptanoate, diethylene glycol diisononanoate; and esters of pentaerythritol such as pentaerythrytyl tetraisostearate;
fatty alcohols which are liquid at room temperature with a branched and/or unsaturated carbon chain having from 12 to 26 carbon atoms such as octyl dodecanol, isostearyl alcohol, 2-butyloctanol, 2-hexyldecanol, 2-undecylpentadecanol, oleyl alcohol;
higher fatty acids such as oleic acid, linoleic acid, linolenic acid;
carbonates;
acetates;
citrates;
fluorinated oils which are optionally partially hydrocarbon- and/or silicone-based, such as fluorosilicone oils, fluorinated polyethers, fluorinated silicones as described in the document EP-A-847752;
silicone oils such as linear or cyclic nonvolatile polydimethylsiloxanes (PDMS); polydimethyl-siloxanes comprising alkyl, alkoxy or phenyl groups which are pendent or at the silicone chain end, groups having from 2 to carbon atoms; phenylated silicones such as phenyl trimethicones, phenyl dimethicones, phenyl trimethylsiloxy diphenyl siloxanes, diphenyl dimethicones, diphenyl methyldiphenyl trisiloxanes, 2-phenylethyl trimethylsiloxysilicates,
mixtures thereof.

The composition according to the invention may comprise an amount of volatile or nonvolatile oil ranging from 1 to 80% by weight relative to the total weight of the composition, for example from 5 to 70% by weight, preferably from 10 to 50% by weight and more preferably still from 15 to 40% by weight.

The composition according to the invention may also comprise at least one wax.

The wax considered in the context of the present invention is in general a lipophilic compound which is solid at room temperature (25° C.), deformable or not, with a reversible solid/liquid change of state, having a melting point greater than or equal to 30° C. and which may be as high as 200° C. and in particular as high as 120° C.

By bringing the wax to the liquid state (melting), it is possible to make it miscible with oils and to form a microscopically homogeneous mixture, but on bringing the temperature of the mixture back to room temperature, recrystallization of the wax in the oils of the mixture is obtained.

In particular, the waxes suitable for the invention may have a melting point greater than or equal to 45° C., and in particular greater than or equal to 55° C.

For the purposes of the invention, the melting temperature corresponds to the most endothermic peak temperature observed in thermal analysis (DSC) as described in the ISO 11357-3 standard; 1999. The melting point of the wax may be measured with the aid of differential scanning calorimetry (DSC), for example the calorimeter sold under the name “MDSC 2920” by the company TA Instruments.

The measurement protocol is the following:

a sample of 5 mg of wax deposited in a crucible is subjected to a first rise in temperature ranging from −20° C. to 100° C., at the heating rate of 10° C./minute, and is then cooled from 100° C. to −20° C. at a cooling rate of 10° C./minute and finally subjected to a second rise in temperature ranging from −20° C. to 100° C. at a heating rate of 5° C./minute. During the second rise in temperature, the variation in the difference in the power absorbed by the empty crucible and by the crucible containing the wax sample is measured as a function of the temperature. The melting point of the compound is the temperature value corresponding to the summit of the peak of the curve representing the variation in the difference in the power absorbed as a function of the temperature.

The waxes which can be used in the compositions according to the invention are chosen from waxes, which are solid at room temperature, of animal, plant, inorganic or synthetic origin, and mixtures thereof.

The waxes which may be used in the compositions according to the invention generally have a hardness ranging from 0.01 MPa to 15 MPa, in particular greater than 0.05 MPa and in particular greater than 0.1 MPa.

The hardness is determined by the measurement of the compression force measured at 20° C. with the aid of the texturometer sold under the name TA-XT2 by the company RHEO, equipped with a stainless steel cylinder 2 mm in diameter and moving at the measuring speed of 0.1 mm/s, and penetrating into the wax to a penetration depth of 0.3 mm.

The measurement protocol is the following:

the wax is melted at a temperature equal to the melting point of the wax +10° C. The molten wax is poured into a container 25 mm in diameter and 20 mm in depth. The wax is recrystallized at room temperature (25° C.) for 24 hours such that the surface of the wax is flat and smooth, and then the wax is stored for at least 1 hour at 20° C. before carrying out the measurement of the hardness or the tackiness.

The spindle of the texturometer is moved at the speed of 0.1 mm/s, and then penetrates into the wax to a penetration depth of 0.3 mm. When the spindle has penetrated into the wax to the depth of 0.3 mm, the spindle is maintained fixed for 1 second (corresponding to the relaxation time) and is then withdrawn at the speed of 0.5 mm/s.
The hardness value is the maximum compression force measured divided by the surface area of the texturometer cylinder in contact with the wax.

By way of illustration of the waxes suitable for the invention, there may be mentioned in particular hydrocarbon waxes such as beeswax, lanolin wax, Chinese insect waxes, rice bran wax, carnauba wax, candelilla wax, ouricury wax, Esparto wax, berry wax, shellac wax, Japan wax, sumac wax, montan wax, orange and lemon waxes, microcrystalline waxes, paraffins and ozokerite, polyethylene waxes, the waxes obtained by Fisher-Tropsch synthesis and waxy copolymers and esters thereof.

There may also be mentioned waxes obtained by catalytic hydrogenation of animal or vegetable oils having linear or branched C₈-C₃₂ fatty chains. Among these, there may be mentioned in particular isomerized jojoba oil such as the trans isomerized partially hydrogenated jojoba oil manufactured or marketed by the company DESERT WHALE under the trade mark Iso-Jojoba-50®, hydrogenated sunflower oil, hydrogenated castor oil, hydrogenated coprah oil, hydrogenated lanolin oil and di(1,1,1-trimethylolpropane) tetrastearate sold under the name Hest 2T-4S® by the company HETERENE.

There may also be mentioned silicone waxes and fluorinated waxes.

It is also possible to use the waxes obtained by hydrogenation of castor oil esterified with cetyl alcohol, which are sold under the names Phytowax ricin 16L64® and 22L73® by the company SOPHIM. Such waxes are described in application FR-A-2792190.

According to a preferred embodiment, the compositions according to the invention comprise at least one wax called tacky wax, that is to say having a tackiness greater than or equal to 0.1 N.s and a hardness of less than or equal to 3.5 MPa.

The tacky wax used may have in particular a tackiness ranging from 0.1 N.s to 10 N.s, in particular ranging from 0.1 N.s to 5N.s, preferably ranging from 0.2 to 5 N.s and even better ranging from 0.3 to 2 N.s.

The tackiness of the wax is determined by measuring the variation of the force (compression force) as a function of time, at 20° C. according to the protocol indicated above for the hardness.

During the relaxation time of 1 s, the force (compression force) decreases greatly until it becomes zero and then, during the withdrawal of the spindle, the force (pulling force) becomes negative and then increases again toward the value 0. The tackiness corresponds to the integral of the curve of the force as a function of time for the portion of the curve corresponding to the negative force values. The tackiness value is expressed as N.s.

The tacky wax which may be used generally has a hardness of less than or equal to 3.5 MPa, in particular ranging from 0.01 MPa to 3.5 MPa, in particular ranging from 0.05 MPa to 3 MPa.

As tacky wax, there may be used a C₂₀-C₄₀ alkyl (hydroxystearyloxy)stearate (the alkyl group comprising from 20 to 40 carbon atoms), alone or as a mixture.

Such a wax is in particular sold under the names “Kester Wax K 82 P®”, “Hydroxypolyester K 82 P®” and “Kester Wax K 80 P®” by the company KOSTER KEUNEN.

In the present invention, it is also possible to use waxes provided in the form of small particles having a size, expressed as mean “effective” diameter by volume D[4,3] of the order of 0.5 to 30 micrometers, in particular 1 to 20 micrometers, and more particularly 5 to 10 micrometers, designated later by the expression “microwaxes”.

The particle sizes may be measured by various techniques, there may be mentioned in particular (dynamic and static) light scattering techniques, Coulter counter methods, measurements based on rate of sedimentation (linked to size via Stokes' law) and microscopy. These techniques make it possible to measure a particle diameter and, for some of them, a particle size distribution.

Preferably, the particle sizes and the particle size distributions of the compositions according to the invention are measured by static light scattering by means of a commercial particle size analyzer of the MasterSizer 2000 type from Malvern. The data are processed on the basis of the Mie scattering theory. This theory, which is accurate for isotropic particles, makes it possible to determine an “effective” particle diameter in the case of nonspherical particles. This theory is described in particular in the book by Van de Hulst, H. C., “Light Scattering by Small Particles”, chapters 9 and 10, Wiley, New York, 1957.

The composition is characterized by its mean “effective” diameter by volume D[4,3], defined in the following manner:

$$D [ 4 , 3 ] = \frac{{\sum }_{i} {V}_{i} \cdot {d}_{i}}{{\sum }_{i} {V}_{i}}$$

where Vᵢ represents the volume of the particles with an effective diameter dᵢ. This parameter is described in particular in the technical documentation for the particle size analyzer.
The measurements are carried out at 25° C., on a dilute dispersion of particles, obtained from the composition in the following manner: 1) dilution by a factor of 100 with water, 2) homogenization of the solution, 3) allowing the solution to rest for 18 hours, 4) recovery of the whitish homogeneous supernatant.
The “effective” diameter is obtained by taking a refractive index of 1.33 for water and a mean refractive index of 1.42 for the particles.

As microwaxes which can be used in the compositions according to the invention, there may be mentioned in particular carnauba microwaxes such as those marketed under the names MicroCare 350° by the company MICRO POWDERS, microwaxes of synthetic wax such as those marketed under the name MicroEase 114S® by the company MICRO POWDERS, the microwaxes consisting of a mixture of carnauba wax and polyethylene wax such as those marketed under the names Micro Care 300® and 310® by the company MICRO POWDERS, microwaxes consisting of a mixture of carnauba wax and synthetic wax such as those marketed under the name Micro Care 325® by the company MICRO POWDERS, polyethylene microwaxes such as those marketed under the names Micropoly 200®, 220®, 220L® and 250S® by the company MICRO POWDERS and polytetrafluoroethylene microwaxes such as those marketed under the names Microslip 519® and 519 L® by the company MICRO POWDERS.

The composition according to the invention may comprise an amount of wax ranging from 0.1 to 40% by weight relative to the total weight of the composition, in particular it may contain an amount of 0.5 to 30%, more particularly of 1 to 20%.

As specified above, the semicrystalline copolymers considered according to the invention can play the role of structuring agents in the composition.

However, the composition may also comprise at least one other structuring agent chosen from thickening agents, the lipophilic gelling agents conventionally used in cosmetics and mixtures thereof.

As lipophilic gelling agents conventionally used in cosmetics, there may be mentioned for example inorganic lipophilic gelling agents such as clays or silicas, polymeric organic lipophilic gelling agents such as partially or totally crosslinked elastomeric organopolysiloxanes, block copolymers of the polystyrene/copoly(ethylene-propylene) type, polyamides and mixtures thereof.

As for the aqueous phase of the composition, it may be thickened with a thickening agent. Among the aqueous phase thickening agents which can be used according to the invention, there may be mentioned cellulosic thickeners, clays, polysaccharides, acrylic polymers, associative polymers and mixtures thereof.

As hydrophilic thickening agent, there may be mentioned in particular the AMPS/acrylamide copolymers of the SEPIGEL® or SIMULGEL® type marketed by the company SEPPIC.

In the composition according to the invention, the amount of aqueous phase thickening agent may range from 0.1% to 15% by weight, relative to the total weight of the composition, preferably from 1% to 10%, and even better from 1 to 5% by weight.

Surfactants

The composition may comprise nonionic, anionic, cationic or amphoteric surfactants or surface-active emulsifiers. Reference may be made to the document “Encyclopedia of Chemical Technology, KIRK-OTHMER”, volume 22, p. 333-432, 3rd edition, 1979, WILEY, for the definition of the properties and functions (emulsifier) of surfactants, in particular p. 347-377 of this reference, for anionic, amphoteric and nonionic surfactants.

Such surfactants may be present in particular in a proportion ranging from 0.1 to 20%, and even better from 0.3% to 15% by weight relative to the total weight of the composition.

According to the invention, an emulsifying surfactant is generally used which is appropriately chosen in order to obtain an oil-in-water emulsion. In particular, it is possible to use an emulsifying surfactant having at 25° C. an HLB balance (hydrophilic-lipophilic balance) according to GRIFFIN, greater than or equal to 8. The HLB value according to GRIFFIN is defined in J. Soc. Cosm. Chem. 1954 (volume 5), pages 249-256.

The surfactants preferably used in the composition according to the invention are chosen:
from nonionic surfactants:

a) nonionic surfactants having an HLB greater than or equal to 8 at 25° C., used alone or as a mixture; there may be mentioned in particular:
oxyethylenated and/or oxypropylenated glycerol ethers which may comprise from 1 to 150 oxyethylene and/or oxypropylene units;
oxyethylenated and/or oxypropylenated ethers (which may comprise from 1 to 150 oxyethylene and/or oxypropylene units) of fatty alcohols, in particular as C₈-C₂₄, and preferably as C₁₂-C₁₈, such as the oxyethylenated ether of stearyl alcohol containing 20 oxyethylene units (CTFA name “Steareth-20”) such as BRIJ 78® marketed by the company UNIQEMA, the oxyethylenated ether of cetearyl alcohol containing 30 oxyethylene units (CTFA name “Ceteareth-30”) and the oxyethylenated ether of the mixture of C₁₂-C₁₅ fatty alcohols comprising 7 oxyethylene units (CTFA name “C₁₂₋₁₅ Pareth-7”) such as that marketed under the name NEODOL 25-7® by SHELL CHEMICALS;
esters of fatty acids, in particular as C₈-C₂₄, and preferably as C₁₆-C₂₂, and of polyethylene glycol (or PEG) (which may comprise from 1 to 150 oxyethylene units), such as PEG-50 stearate and PEG-40 monostearate marketed under the name MYRJ 52P® by the company UNIQEMA;
esters of fatty acids, in particular as C₈-C₂₄, and preferably as C₁₆-C₂₂, and of oxyethylenated and/or oxypropylenated glycerol ethers (which may comprise from 1 to 150 oxyethylene and/or oxypropylene units), such as polyoxyethylenated glyceryl monostearate containing 200 oxyethylene units, sold under the name Simulsol 220TM® by the company SEPPIC; polyoxyethylenated glyceryl stearate containing 30 oxyethylene units such as the product TAGAT S® sold by the company GOLDSCHMIDT, polyoxyethylenated glyceryl oleate containing 30 oxyethylene units such as the product TAGAT O® sold by the company GOLDSCHMIDT, polyoxyethylenated glyceryl cocoate containing 30 oxyethylene units such as the product VARIONIC LI 13® sold by the company SHEREX, polyoxyethylenated glyceryl isostearate containing 30 oxyethylene units such as the product TAGAT L® sold by the company GOLDSCHMIDT and polyoxyethylenated glyceryl laurate containing 30 oxyethylene units such as the product TAGAT I® by the company GOLDSCHMIDT;
esters of fatty acids, in particular as C₈-C₂₄, and preferably as C₁₆-C₂₂, and of oxyethylenated and/or oxypropylenated sorbitol ethers (which may comprise from 1 to 150 oxyethylene and/or oxypropylene units), such as polysorbate 60 sold under the name Tween 60® by the company UNIQEMA;
dimethicone copolyol, such as the product sold under the name Q2-5220® by the company DOW CORNING;
dimethicone copolyol benzoate such as that sold under the name FINSOLV SLB 101® and 201® by the company FINTEX;
copolymers of propylene oxide and ethylene oxide, also called EO/PO polycondensates,
and mixtures thereof.

The EO/PO polycondensates are more particularly copolymers consisting of polyethylene glycol and polypropylene glycol blocks, such as for example the polyethylene glycol/polypropylene glycol/polyethylene glycol triblock polycondensates. These triblock polycondensates have for example the following chemical structure:

H—(O—CH₂—CH₂)ₐ—(O—CH(CH₃)—CH₂)_{b}—(O—CH₂—CH₂)ₐ—OH,

in which formula a ranges from 2 to 120, and b ranges from 1 to 100.

The EO/PO polycondensates preferably have a weight-average molecular mass ranging from 1000 to 15 000, and even better ranging from 2000 to 13 000. Advantageously, said EO/PO polycondensates have a cloud temperature, at 10 g/l in distilled water, greater than or equal to 20° C., preferably greater than or equal to 60° C. The cloud temperature is measured according to the ISO 1065 standard. As EO/PO polycondensate which can be used according to the invention, there may be mentioned the polyethylene glycol/polypropylene glycol/polyethylene glycol triblock polycondensates sold under the names SYNPERONIC® such as SYNPERONIC PE/L44® and SYNPERONIC PE/F127® by the company ICI;

b) nonionic surfactants having an HLB of less than 8 at 25° C., optionally combined with one or more nonionic surfactants having an HLB greater than 8 at 25° C., as cited above, such as:
esters and ethers of monosaccharides such as sucrose stearate, sucrose cocoate, sorbitan stearate and mixtures thereof, for example Arlatone 2121® sold by the company ICI or SPAN 65V from the company UNIQEMA;
esters of fatty acids, in particular as C₈-C₂₄, and preferably as C₁₆-C₂₂, and of polyol, in particular of glycerol and sorbitol, such as glyceryl stearate, for example sold under the name TEGIN M® by the company GOLDSCHMIDT, glyceryl laurate such as the product sold under the name IMWITOR 312® by the company HULS, polyglyceryl-2 stearate, sorbitan tristearate and glyceryl ricinoleate;
oxyethylenated and/or oxypropylenated ethers such as the oxyethylenated ether of stearyl alcohol containing 2 oxyethylene units (CTFA name “Steareth-2”) such as BRIJ 72® marketed by the company UNIQEMA;
the cyclomethicone/dimethicone copolyol mixture sold under the name Q2-3225C® by the company DOW CORNING;
from anionic surfactants:

a) salts of polyoxyethylenated fatty acids, in particular amino salts or alkali metal salts, and mixtures thereof;

b) salts of C₁₆-C₃₀ fatty acids, in particular amino salts such as triethanolamine stearate or 2-amino-2-methylpropane-1,3-diol stearate;

c) phosphoric esters and their salts such as “DEA oleth-10 phosphate” (Crodafos N 10N® from the company CRODA) or monopotassium monocetyl phosphate (Amphisol K® from Givaudan or ARLATONE MAP 160K® from the company UNIQUEMA);

d) sulfosuccinates such as “Disodium PEG-5 citrate lauryl sulfosuccinate” and “Disodium ricinoleamido MEA sulfosuccinate”;

e) alkyl ether sulfates such as sodium lauryl ether sulfate;

f) isethionates;

g) acyl glutamates such as “Disodium hydrogenated tallow glutamate” (AMISOFT HS-21 R® marketed by the company AJINOMOTO) and mixtures thereof,
from cationic surfactants:

a) alkyl-imidazolidiniums such as isostearylethylimidonium etho-sulfate;

b) ammonium salts such as (C12-30 alkyl)-tri(C₁₋₄ alkyl)ammonium halides such as N,N,N-trimethyl-1-docosanaminium chloride (or behentrimonium chloride),
from amphoteric surfactants: N-acyl-amino acids such as N-alkyl-amino acetates and disodium cocoamphodiacetate, and amine oxides such as stearamine oxide, or silicone surfactants such as dimethicone copolyol phosphates such as that sold under the name PECOSIL PS 100® by the company PHOENIX CHEMICAL.

According to one embodiment, the composition according to the invention comprises, as emulsifying system, the following combination:
of at least one C₁₀-C₃₀ alkyl phosphate surfactant, and
of at least one C₈-C₂₄ fatty alcohol ether of polyethylene glycol, said ether comprising from 1 to 19 oxyethylene units and having an HLB <8 at 25° C.

According to one embodiment, said emulsifying system may additionally comprise at least one C₈-C₂₄ fatty alcohol ether of polyethylene glycol, said ether comprising from 20 to 1000 oxyethylene units and having an HLB >8 at 25° C.

Film-Forming Polymer

The composition according to the invention may additionally comprise at least one film-forming polymer.

The expression “film-forming” polymer is understood to mean a polymer capable of forming, on its own or in the presence of a film-forming aid, a continuous and adherent film on a support, in particular on the keratin materials.

The film-forming polymer may be dispersed in the form of solid particles in an aqueous phase of the composition or alternatively solubilized or dispersed in the form of solid particles in a liquid fatty phase. The composition may comprise a mixture of these polymers. When the film-forming polymer is in the form of solid particles, these particles may have a mean particle size ranging from 5 nm to 600 nm, and preferably from 20 nm to 300 nm.

The film-forming polymer may be present in the composition according to the invention in a dry matter content ranging from 0.1% to 60% by weight relative to the total weight of the composition, preferably from 0.5% to 40% by weight, and even better from 1% to 30% by weight.

Among the film-forming polymers which can be used in the composition of the present invention, there may be mentioned synthetic polymers, of the free-radical type or of the polycondensate type, polymers of natural origin, and mixtures thereof.

The expression free-radical film-forming polymer is understood to mean a polymer obtained by polymerization of monomers with in particular ethylenic unsaturation, each monomer being capable of homopolymerizing (by contrast to the polycondensates).

The free-radical type film-forming polymers may be in particular vinyl polymers or copolymers, in particular acrylic polymers.

The vinyl film-forming polymers may result from the polymerization of ethylenically unsaturated monomers having at least one acid group and/or esters of these acid monomers and/or amides of these acid monomers.

As a monomer bearing an acid group, it is possible to use unsaturated α,β-ethylenic carboxylic acids such as acrylic acid, methacrylic acid, crotonic acid, maleic acid, itaconic acid. Use is preferably made of (meth)acrylic acid and crotonic acid, and more particularly (meth)acrylic acid.

The esters of acid monomers are advantageously chosen from the esters of (meth)acrylic acid (also called (meth)acrylates), especially alkyl, in particular C₁-C₃₀ alkyl, preferably C₁-C₂₀, (meth)acrylates, aryl, in particular C₆-C₁₀ aryl, (meth)acrylates, hydroxyalkyl, in particular C₂-C₆ hydroxyalkyl, (meth)acrylates.

Among the alkyl (meth)acrylates, there may be mentioned methyl methacrylate, ethyl methacrylate, butyl methacrylate, isobutyl methacrylate, 2-ethylhexyl methacrylate, lauryl methacrylate, cyclohexyl methacrylate.

Among the hydroxyalkyl (meth)acrylates, there may be mentioned hydroxyethyl acrylate, 2-hydroxypropyl acrylate, hydroxyethyl methacrylate, 2-hydroxypropyl methacrylate.

Among the aryl (meth)acrylates, there may be mentioned benzyl acrylate and phenyl acrylate.

The (meth)acrylic acid esters particularly preferred are alkyl (meth)acrylates.

The alkyl group of the esters may be either fluorinated or perfluorinated, that is to say that some or all of the hydrogen atoms of the alkyl group are substituted with fluorine atoms.

As amides of acid monomers, there may be mentioned for example (meth)acrylamides, and in particular N-alkyl, in particular C₂-C₁₂ alkyl, (meth)acrylamides. Among the N-alkyl(meth)acrylamides, there may be mentioned N-ethylacrylamide, N-t-butylacrylamide, N-t-octylacrylamide and N-undecylacrylamide.

The vinyl film-forming polymers may also result from the homopolymerization or copolymerization of monomers chosen from the vinyl esters and the styrene monomers. In particular, these monomers may be polymerized with acid monomers and/or their esters and/or their amides, such as those mentioned above.

As example of vinyl esters, there may be mentioned vinyl acetate, vinyl neodecanoate, vinyl pivalate, vinyl benzoate and vinyl t-butyl benzoate.

As styrene monomers, there may be mentioned styrene and alpha-methylstyrene.

It is possible to use any monomer known to a person skilled in the art entering into the categories of acrylic and vinyl monomers (including the monomers modified by a silicone chain).

Among the film-forming polycondensates, there may be mentioned polyurethanes, polyesters, polyester amides, polyamides, epoxy ester resins, polyureas.

The polyurethanes may be chosen from anionic, cationic, nonionic or amphoteric polyurethanes, polyurethanes-acrylics, polyurethanes-polyvinylpyrrolidones, polyester-polyurethanes, polyureas, polyurea-polyurethanes, and mixtures thereof.

The optionally modified polymers of natural origin may be chosen from shellac resin, sandarac gum, dammars, elemis, copals, cellulose polymers, and mixtures thereof.

According to a first embodiment, the additional film-forming polymer may be present in the form of particles in aqueous dispersion, generally known by the name of latex or pseudolatex. The techniques for preparing these dispersions are well known to persons skilled in the art.

As aqueous dispersion of film-forming polymer, it is possible to use the acrylic dispersions sold under the names NEOCRYL XK-90°, NEOCRYL A-1070°, NEOCRYL A-1090°, NEOCRYL BT-62°, NEOCRYL A-1079°, NEOCRYL A-523° by the company DSM NEORESINS, DOW LATEX 432® by the company DOW CHEMICAL, DAITOSOL 5000 AD® by the company DAITO KASEY KOGYO; SYNTRAN 5760, SYNTRAN 5190 and SYNTRAN 5170 marketed by the company INTERPOLYMER or alternatively the aqueous dispersions of polyurethane sold under the names NEOREZ R-981°, NEOREZ R-974® by the company DSM NEORESINS, AVALURE UR-405°, AVALURE UR-410°, AVALURE UR-425°, AVALURE UR-450°, SANCURE 875®, SANCURE 861®, SANCURE 878®, SANCURE 2060® by the company NOVEON, IMPRANIL 85® by the company BAYER, AQUAMERE H-1511° by the company HYDROMER.

As aqueous dispersions of film-forming polymer, it is also possible to use the dispersions of polymers resulting from the free-radical polymerization of one or more free-radical monomers inside and/or partially at the surface, of preexisting particles of at least one polymer chosen from the group consisting of polyurethanes, polyureas, polyesters, polyester amides and/or alkyds. These polymers are generally called hybrid polymers.

According to another variant embodiment of the composition according to the invention, the film-forming polymer may be present in a liquid fatty phase comprising organic oils or solvents.

The expression “liquid fatty phase” is understood to mean, for the purposes of the invention, a fatty phase that is liquid at room temperature (25° C.) and atmospheric pressure (760 mmHg, that is 10⁵ Pa), composed of one or more fatty substances that are liquid at room temperature, also called oils, generally mutually compatible. Preferably, the liquid fatty phase comprises a volatile oil, optionally mixed with a nonvolatile oil, it being possible for the oils to be chosen from the oils mentioned above.

According to another embodiment of the composition according to the invention, the film-forming polymer may be present in the form of surface-stabilized particles dispersed in the liquid fatty phase.

The dispersion of surface-stabilized polymer particles may be prepared as described in the document EP-A-749747.

The polymer particles may be surface-stabilized by means of a stabilizer which may be a block polymer, a graft polymer and/or a random polymer, alone or as a mixture.

Film-forming polymer dispersions in the liquid fatty phase, in the presence of stabilizing agents, are in particular described in the documents EP-A-749746, EP-A-923928, EP-A-930060.

The size of the polymer particles in dispersion, either in the aqueous phase or in the liquid fatty phase, may range from 5 nm to 600 nm, and preferably from 20 nm to 300 nm.

According to another embodiment of the composition according to the invention, the film-forming polymer may be solubilized in the liquid fatty phase, the film-forming polymer is then said to be a fat-soluble polymer.

By way of example of fat-soluble polymer, there may be mentioned copolymers of vinyl ester (the vinyl group being directly linked to the oxygen atom of the ester group and the vinyl ester having a linear or branched, saturated hydrocarbon radical having from 1 to 19 carbon atoms, linked to the carbonyl of the ester group) and of at least one other monomer which may be a vinyl ester (different from the vinyl ester already present), an α-olefin (having from 8 to 28 carbon atoms), an alkyl vinyl ether (in which the alkyl group comprises from 2 to 18 carbon atoms), or an allyl or methallyl ester (having a linear or branched, saturated hydrocarbon radical having from 1 to 19 carbon atoms, linked to the carbonyl of the ester group).

These copolymers may be crosslinked with the aid of crosslinking agents which may be either of the vinyl type, or of the allyl or methallyl type, such as tetraallyloxyethane, divinylbenzene, divinyl octanedioate, divinyl dodecanedioate and divinyl octadecanedioate.

As examples of these copolymers, there may be mentioned the copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethyl-propionate/vinyl stearate, allyl dimethylpropionate/vinyl stearate, vinyl propionate/vinyl stearate, crosslinked with 0.2% of divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene crosslinked with 0.2% divinylbenzene and allyl propionate/allyl stearate crosslinked with 0.2% divinylbenzene.

As fat-soluble film-forming polymers, there may also be mentioned fat-soluble homopolymers, and in particular those resulting from the homopolymerization of vinyl esters having from 9 to 22 carbon atoms or alkyl acrylates or methacrylates, alkyl radicals having from 10 to 20 carbon atoms.

Such fat-soluble homopolymers may be chosen from polyvinyl stearate, polyvinyl stearate crosslinked with divinylbenzene, diallyl ether or diallyl phthalate, polystearyl (meth)acrylate, polyvinyl laurate, polylauryl (meth)acrylate, it being possible for these poly(meth)acrylates to be crosslinked with the aid of ethylene glycol or tetraethylene glycol dimethacrylate.

The fat-soluble copolymers and homopolymers defined above are known and are described in particular in application FR-A-2262303; they may have a weight-average molecular weight ranging from 2000 to 500 000 and preferably from 4000 to 200 000.

As fat-soluble film-forming polymers which can be used in the invention, there may also be mentioned polyalkylenes and in particular copolymers of C₂-C₂₀ alkenes, such as polybutene, alkylcelluloses with a linear or branched, saturated or unsaturated, C₁ to C₈ alkyl radical such as ethylcellulose and propylcellulose, copolymers of vinylpyrolidone (abbreviated VP hereinafter) and in particular copolymers of vinylpyrrolidone and of a C₂ to C₄₀, and even better C₃ to C₂₀, alkene. By way of example of VP copolymer which can be used in the invention, there may be mentioned the VP/vinyl acetate, VP/ethyl methacrylate, butylated polyvinylpyrolidone (PVP), VP/ethyl methacrylate/methacrylic acid, VP/eicosene, VP/hexadecene, VP/triacontene, VP/styrene, VP/acrylic acid/lauryl methacrylate copolymer.

The composition according to the invention may comprise a film-forming aid promoting the formation of a film with a film-forming polymer. Such a film-forming agent may be chosen from all the compounds known to persons skilled in the art as being capable of playing the desired role, and in particular may be chosen from plasticizers and coalescing agents.

Other Additives Including Fillers

The composition of the invention may additionally comprise any additive customarily used in cosmetics, such as fillers, antioxidants, preservatives, perfumes, neutralizers, plasticizers, cosmetic agents such as for example emollients, moisturizers, vitamins, sunscreens, and mixtures thereof. These additives may be present in the composition in an amount ranging from 0.01 to 30%, of the total weight of the composition.

The fillers may be chosen from those well known to a person skilled in the art and commonly used in cosmetic compositions. The fillers may be inorganic or organic, lamellar or spherical. There may be mentioned talc, mica, silica, kaolin, polyamide powders such as Nylon or Orgasol® from Arkema, poly-β-alanine and polyethylene, powders of tetrafluoroethylene polymers, such as Teflon®, lauroyl lysine, starch, boron nitride, expanded polymeric hollow microspheres such as those of polyvinylidene chloride/acrylonitrile such as ExpanceL® (Nobel Industrie), acrylic powders such as Polytrap® (Dow Corning), polymethyl methacrylate particles and microbeads of silicone resin (Tospearls® from Toshiba, for example), precipitated calcium carbonate, magnesium carbonate and hydrocarbonate, hydroxyapatite, hollow silica microspheres (SILICA BEADS® from MAPRECOS), glass or ceramic microcapsules, metal soaps derived from organic carboxylic acids having from 8 to 22 carbon atoms, preferably from 12 to 18 carbon atoms, for example zinc, magnesium or lithium stearate, zinc laurate, magnesium myristate.

The fillers may represent from 0.1 to 25%, and even better from 1 to 20%, by weight of the total weight of the composition.

Of course persons skilled in the art will be careful to choose the optional additional additives and/or their quantity such that the advantageous properties of the composition according to the invention are not impaired by the addition envisaged.

The composition according to the invention may be prepared by known methods, generally used in the cosmetic field.

The composition according to the invention is preferably intended to be heated.

The heating of the composition, in particular in order to curl the eyelashes, may be carried out after its application, by means, for example, of devices as described in patent U.S. Pat. No. 5,853,010.

The composition according to the invention may be packaged in a packaging and application set (1) comprising:

i) a reservoir (2);

ii) a device (10) for applying said cosmetic composition according to the invention; and

iii) heating means (53) in order to bring said composition, simultaneously with or subsequent to its application, to a temperature greater than the melting point of the semicrystalline polyurethane/polyester copolymer which it contains.

In particular, another subject of the invention is a set for the packaging and application (1) of a makeup and/or care composition, in particular for the eyelashes and/or the eyebrows, comprising:

i) a reservoir (2);

ii) a makeup and/or care composition placed inside the reservoir (2), said composition comprising, in a physiologically acceptable medium, an effective quantity of at least one semicrystalline polyurethane/polyester copolymer,

iii) a device (10) for applying the makeup and/or care composition; and

iv) heating means (53) for bringing said composition, simultaneously with or subsequent to its application, to a temperature greater than the melting point of the semicrystalline polyurethane/polyester copolymer which it contains.

According to one embodiment, the heating means are formed by a device separate from the application device or part, the set being designed in the form of a packaging and application device additionally comprising a container containing a composition in accordance with the invention. Such a device may be packaged inside a blister-type packaging. The heating means may be of the type described in patents U.S. Pat. No. 6,009,884 or U.S. Pat. No. 5,853,010. Other devices designed in the form of heating tweezers (in the case of the eyelashes) may also be used. Such devices are described in particular in patent U.S. Pat. No. 6,220,252.

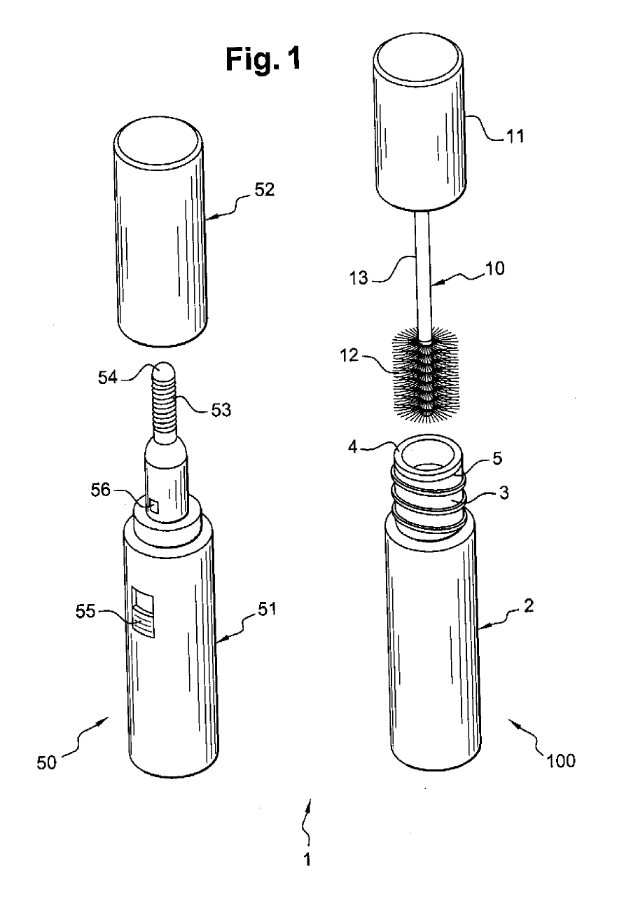

The kit 1 represented in FIG. 1 comprises a set (100) for packaging and applying the mascara and a heating device (50), separated from said packaging and application set.

The two devices (100) and (50) may be sold together in the same packaging, of the blister type, or separately.

The unit (100) containing the mascara may be sold separately. The packaging and application set (100) comprises a container (2), comprising the composition according to the invention, surmounted by a threaded neck (3) whose free edge delimits an opening (4). A draining part (5), is mounted in the opening (4). The set (100) also comprises an application device (10) comprising a stopper (11) integrally attached to a shaft (13) whose end comprises an applicator (12), generally designed in the form of an arrangement of fibers maintained between the two branches of a twisted iron wire. An inner surface of the stopper (11) is threaded so as to cooperate with the thread of the neck (3). Thus, when the applicator (12) and the shaft (13) are placed inside the container (2), the thread of the stopper (11) engages with the thread of the neck (3) such that the stopper closes the opening (4) of the container in a leaktight manner. Such packaging and application sets are well known.

The heating device (50) is in accordance with what is described in patent U.S. Pat. No. 6,009,884. It mainly comprises a gripping part (51) and a cap (52). A battery is placed inside the gripping part (51) and is connected to a heating wire (53) designed in the form of a helical coil placed on a shaft (54). A switch (55) makes it possible to switch the device on or respectively to switch it off. A diode (56), when it changes color, indicates that the device is at the required temperature, and that it is therefore ready to be used.

The power supply to the heating part via the battery is 12 V. The power dissipated is about 1 Watt. The heating wire (53) may be made of a nickel/chromium alloy.

According to this embodiment, the mascara is applied in the cold state in a conventional manner on the eyelashes by means of a brush (12) and then heated after application: the user engages the heating part (53) of the device (50) with the eyelashes so as to bring the deposited product to the melting temperature of the composition.

Upon cooling, the composition returns to its semicrystalline state very rapidly because of the small width of the melting peak. The eyelashes are set in their desired curl configuration in a lasting manner.

The invention is illustrated in greater detail in the following examples and by FIG. 1.

### EXAMPLES

The protocol for formulating the two mascara compositions presented below is the “conventional” protocol for mascaras by phase inversion. It comprises in particular the steps consisting in heating the fatty phase to 98° C. and in adding the aqueous phase, heated beforehand to 93° C., with vigorous stirring.

The polymer, used here in the form of an aqueous dispersion, is incorporated at the end of the formulation, when the temperature of the composition is close to 40° C.

### Composition No. 1:

| | |
|---|---|
| beeswax | 7.4% |
| carnauba wax | 3.5% |
| iron oxide | 7% |
| Simulgel 600 ® | 3.5% |
| Disperbond ® D31W40** | 8%* |
| (Disperbond ® D31S as a 40% aqueous dispersion) | |
| PEG-200 ® glyceryl stearate | 4% |
| antifoam | 0.1% |
| preservatives | qs |
| water | qs 100 |

*% expressed by weight of copolymer
**polycaprolactone/4,4′-diphenylmethane diisocyanate copolymer

### Composition No. 2:

| | |
|---|---|
| beeswax | 7.4% |
| carnauba wax | 3.5% |
| iron oxide | 7% |
| Simulgel 600 ® | 3.5% |
| Disperbond ® D39W50** | 10%* |
| (Disperbond ® D39S as a 50% aqueous dispersion) | |
| PEG-200 ® glyceryl stearate | 4% |
| antifoam | 0.1% |
| preservatives | qs |
| water | qs 100 |

*% expressed by weight of copolymer
**polycaprolactone/4,4′-diphenylmethane diisocyanate copolymer

After application of each of these compositions to the eyelashes and heating the film of composition for a few seconds with the aid of a heating brush, these mascaras were judged to have good staying power and to allow significant improvement in the curling of the eyelashes.

1. A cosmetic composition comprising, in a physiologically acceptable medium, an effective quantity of at least one semicrystalline polyurethane/polyester copolymer and at least one dye substance, said copolymer having a melting point greater than or equal to 20° C.

2. The composition as claimed in claim 1, wherein said copolymer has a melting point ranging from 25° C. to 100° C.

3. The composition as claimed in claim 1, wherein said copolymer is obtained by a reaction between at least one crystalline or semicrystalline aliphatic polyester with alpha- and omega-OH ends and at least one aliphatic, cycloaliphatic or aromatic C3-C50 diisocyanate derivative.

4. The composition as claimed in claim 3, wherein the diisocyanate derivative is of formula
OCN—R₅—NCO
wherein R5 is a divalent group chosen from alkylene, arylene, cycloalkylene, alkarylene or arylalkylene groups, said groups being linear or branched and comprising from 1 to 50 carbon atoms and it being possible for said groups to comprise at least one oxygen, sulfur and/or nitrogen atom.

5. The composition as claimed in claim 3, in which the isocyanate derivative is chosen from toluene diisocyanate (TDI), 4,4′ diphenylmethane diisocyanate (MDI), naphthalene diisocyanate, phenylene diisocyanate, xylene diisocyanate (XDI), tetramethylene xylene diisocyanate (TMXDI), isophorone diisocyanate (TPDI), 4,4′ dicyclohexylmethane diisocyanate (HMDI) and hexamethylene diisocyanate (HDI).

6. The composition as claimed in claim 1, wherein the polyester block(s) forming said copolymer result from the polyaddition of a C3-20 lactone substituted or unsubstituted with a C1-6 alkyl group.

7. The composition as claimed in claim 6, wherein said lactone is chosen from β-propiolactone; β-butyrolactone; β-valerolactone; δ-valerolactone; δ-caprolactone; ε-caprolactone; ω-enantholactone; ω-caprylolactone; ω-laurolactone.

8. The composition as claimed in claim 8, wherein said lactone is ε-caprolactone.

9. The composition as claimed in claim 1, wherein said copolymer is derived from the reaction of at least one polycaprolactone with at least 4,4′ diphenylmethane diisocyanate (MDI) or isophorone diisocyanate (IPDI).

10. The composition as claimed in claim 1, wherein the polyester(s) is/are obtained by polycondensation between at least one linear, branched or cyclic, preferably linear, C4-50 aliphatic diacid and at least one linear, substituted or cyclic, preferably linear, aliphatic C4-C50 diol.

11. The composition as claimed in claim 1, wherein the semicrystalline polyurethane/polyester copolymer is obtained by a reaction carried out in the presence of a coupling agent chosen from dials, diamine derivatives or amino alcohol derivatives.

12. The composition as claimed in claim 1, comprising from 1 to 60% by weight, of semicrystalline polyurethane/polyester copolymer(s).

13. The composition as claimed in claim 1, which comprises an aqueous phase.

14. The composition as claimed in claim 13, wherein the aqueous phase represents from 5% to 95% by weight, relative to the total weight of the composition.

15. The composition as claimed in claim 13, wherein the aqueous phase is thickened with at least one thickening agent.

16. The composition as claimed in claim 1, which comprises at least one film-forming polymer.

17. The composition as claimed in claim 16, wherein the film-forming polymer is present in a dry matter content ranging from 0.1% to 60% by weight relative to the total weight of the composition.

18. The composition as claimed in claim 1, wherein the dye substance is present in an amount ranging from 0.1% to 20% by weight, relative to the total weight of the composition.

19. The composition as claimed in claim 1, which is a composition for coating keratin fibers.

20. A cosmetic method for making up or for the nontherapeutic care of the keratin fibers comprising the application, to the keratin fibers, of a cosmetic composition as claimed in claim 1, said composition being, prior to, simultaneously with or subsequent to its application, heated to a temperature greater than or equal to the melting point of the copolymer contained in said composition.

21. A set for the packaging and application of a makeup and/or care composition, in particular for the eyelashes and/or the eyebrows, comprising:
i) a reservoir;
ii) a makeup and/or care composition placed inside the reservoir, said composition comprising, in a physiologically acceptable medium, an effective quantity of at least one semicrystalline polyurethane/polyester copolymer,
iii) a device for applying the makeup and/or care composition; and
iv) heating means for bringing said composition, simultaneously with or subsequent to its application, to a temperature greater than the melting point of said copolymer which it contains.

22. The set as claimed in claim 21, wherein the composition further comprises at least one dye substrate, and the copolymer has a melting point greater than or equal to 20° C.
